(19) United States
(12) Patent Application Publication
(10) Pub. No.: US 2016/0073931 A1
(43) Pub. Date: Mar. 17, 2016

(54) FOOT MORPHOMETRIC MEASURING DEVICE

(71) Applicant: UTI LIMITED PARTNERSHIP, Calgary (CA)

(72) Inventors: REED FERBER, CALGARY (CA); SEAN OSIS, CALGARY (CA)

(73) Assignee: UTI LIMITED PARTNERSHIP, CALGARY, AB (CA)

(21) Appl. No.: 14/888,041

(22) PCT Filed: Apr. 23, 2014
(86) PCT No.: PCT/CA2014/000381

§ 371 Date: Oct. 29, 2015

Related U.S. Application Data
(60) Provisional application No. 61/816,951, filed on Apr. 29, 2013.

(51) Int. Cl. A61B 5/107; A61B 5/00

(52) U.S. Cl. CPC A61B 5/1074; A61B 5/6829; A61B 5/1072

(57) ABSTRACT

A foot measuring device and method measure foot last/curvature, foot width, foot length, arch height, foot volume, first metatarsophalangeal (MTP) joint flexibility and standing rear foot pronation. The foot measuring device includes a base having a heel abutment member, a foot size and alignment graphic, an arch height measurement system, and a MTP joint flexibility measurement lever.

### FIELD OF THE INVENTION

The invention relates to a foot measuring device and method for the purpose of fitting shoes based on the volume and shape of a foot. Specifically the device and method measure foot volume based on arch height, first metatarsal-phalangeal joint flexibility, standing rear foot pronation, and foot length, width and curvature.

### BACKGROUND OF THE INVENTION

In 1925, Charles F. Brannock invented a foot-measuring device that is known throughout the world as The Brannock™. As is well known, The Brannock™ device is the most common device for computing a person's shoe size. That is, almost every shoe store in the world uses The Brannock™ and very few competing devices have since been developed. However, The Brannock™ is based on sizes that are not used by the industry leaders in shoe manufacturing such as Nike™, Adidas™, and New Balance™. Thus, having one's foot measured, for either length or width, using this device is generally not accurate for reasons as explained below. In addition market research has also demonstrated that many shoe stores simply remove the components on The Brannock™ that are necessary for measuring foot width since they are highly inaccurate and not representative of current shoe models. Most importantly, The Brannock™ does not take into consideration an important component of shoe fitting, namely the curvature (or last) of the foot.

FIG. 1 shows foot last and its effect on shoe fit. As shown in (A) of this Figure, it can be seen that a straight-lasted foot 20 is correctly placed inside of a straight-lasted shoe 22 and thus proper fit is achieved. However, (B) shows that the same foot 20 is then placed inside of a curve-lasted shoe 24 and greater contact along the lateral (outside) 20a of the foot with the shoe is achieved while the medial (inside) 20b of the foot has unnecessary room inside the shoe. As shown in (C) of FIG. 1, a curve-lasted foot 26 is correctly placed inside a curve-lasted shoe 24 and proper fit is achieved on the lateral aspect of the foot 20a (indicated by the arrows) whereas insufficient curvature of the shoe allows for improper fit, as shown in (D) (furthest to the right). Foot measurement devices do not generally provide information on the last of the foot even though every shoe, and every shoe model, is built with specific curvatures (lasts) to optimize fit and comfort.

Other more complex devices have also been employed for shoe fitting, including x-ray fluoroscopes in the 1920s; the Clarks™ automatic foot-measuring machine of the 1970s; and high-tech, high-cost 3D laser scanning systems. Simpler measuring systems also exist and include measuring sticks and downloadable printed charts. However these devices do not typically provide a comprehensive overview of foot morphology (length, width, last), nor do they provide overarching information about specific clinical measurements that provide insight into the mechanical behaviour of the foot during dynamic gait.

Numerous studies have been published showing the differences people have in foot last, arch height and big toe flexibility. Goonetilleke et al. from the Hong Kong University of Science and Technology, published a study in 1999 investigating foot last measurements for 50 Hong Kong Chinese participants¹. The foot outlines of the participants were traced and the axes of the heel and the forefoot were determined. Participants were found to have a mean inflare (inward curvature) of 3.2° (SD=1.7° max=5.5° min=0.6°). Moreover, several studies²⁻⁴ have reported sex-specific differences in foot morphology that need to be considered for optimal footwear prescription.

With respect to measuring arch height, it has been reported that visual assessment of the plantar surface is not accurate for determining low and high foot arches (Swedler, Knapik and Grier, 2010)⁶. In contrast, the arch height index (AHI) has been shown to be a valid and reliable method of measuring anatomical aspects of the arch (Butler, Davis and Hamill, 2006)⁶. The measure of AHI is unitless and is defined as the ratio of dorsum height at 50% of total foot length, divided by the foot length from the back of the heel to the head of the first metatarsal, defined as the truncated foot length (Fields et al. 2010)⁷. A low AHI value would indicate a pes planus structure whereas a high AHI value would indicate a pes cavus foot structure. McPoil et al. (2009)⁸ used a similar approach by measuring arch height with a digital caliper and reported that the average amount of arch deformation during weight-bearing, as compared with sitting measures, was between 1.19 cm and 1.35 cm for both males and females.

Testing for Hallux Metatarsophalangeal (MTP) Extension (big toe flexibility) is routine for therapeutic measurement. Manual assessment is documented in, for example in the book Therapeutic measurement and testing: the basics of ROM, MMT, posture, and gait analysis by Lisa Jennings Weaver and Amanda L. Ferg, wherein MTP extension is graded on a qualitative scale from Grade 0 (inflexible) to Grade 5 (normal).⁹ Quantitative measurement using a goniometer is also defined with normal Range of Motion (ROM) as 0-70°.

Hypo- and hypermobility of MTP extension has been implicated in the literature as a primary cause of mechanical foot problems as a result of compensatory movements. For example, Cornwall et al. (2006)¹⁰ reported that reduced MTP extension resulted in significantly more rearfoot eversion motion when walking as compared to individuals with either normal or excessive MTP extension. Thus, adequate range of motion of the MTP joint should be considered when fitting a shoe designed to control foot motion.

### Diabetic Foot

Patients with diabetes or other forms of neuropathy or vascular compromise are generally very sensitive to foot injury. Diabetic foot ulcers are common in diabetic patients and the ulcers often heal poorly, leading to further complications in fitting shoes for such patients. Specialist footwear, custom fitted footwear, or well-fitted athletic shoes are generally recommended for diabetic patients with foot ulcers. Improper shoe fit can result in injury to the foot.

In non-diabetics, simple abrasions and blisters can form when the foot-shape and shoe-shape do not correspond, as a result of atypical contact pressures and forces. For example, runners commonly develop “blackened toes,” callus formation, and blisters during recreational runs as a result of the foot not fitting properly within the shoe. For diabetics, nearly half will develop some degree of peripheral neuropathy (loss of sensation to their feet), and approximately 15% of all diabetic patients will develop a foot ulcer in their lifetime as a result of not being able to “feel” the atypical contact pressures and forces experienced when walking. However, if the shoe were properly fitted to the shape of the foot, the potential for atypical contact pressure and force generation is significantly reduced. For example, in 2007 Harrison and colleagues¹¹ published a study where they measured 100 diabetics for proper shoe fit and concluded that many patients with diabetes wear shoes that do not fit, particularly, shoes that are too narrow for their foot width.

As a result, there is a need for a device and method for determining foot length, width and last in order to provide better fitting shoes for the general population and for patients with specialized needs such as diabetic patients. Specifically there is a need for a device that enables the measurement of each of foot last, arch deformation and first ray (hallux) flexibility.

### PRIOR ART

A review of the prior art reveals that such a system has not been developed. For example, U.S. Pat. No. 2,327,254 to Fisher et al. describes a mechanical foot measuring device that determines foot flare. The device includes a heel cup, a pivotable forefoot platform, and edge pieces that adjust to either side of the ball of the foot. As the edge pieces are adjusted, the forefoot platform rotates to align with the axis of the forefoot. The platform has a pointer which then shows whether the foot requires a straight last, an inflare last or an outflare last.

U.S. Pat. No. 5,123,169 to White et al. describes a foot sizing method encompassing a measurement sequence that uses a tape measure to calculate various foot shape metrics such as foot curvature.

U.S. Pat. No. 5,361,133 to Brown et al. describes a complex foot measurement system using pressure sensors and optical sensors to determine foot length, foot width, shoe size, foot volume, foot shape, force distribution, pronation, arch type, and recommended last type.

U.S. Pat. Nos. 7,757,325 and 7,992,243 to Cook et al. describe a system for custom fitting shoes using a foot measurement device, an adjustable shoe and an infrared activation chamber for heat molding parts of the shoe for custom-fitting. Cook et al. describes the use of the Brannock™ device or a scanning device for taking foot measurements.

There continues to be a need for a relatively simple and inexpensive device and method that allows an operator to quickly, accurately and directly measure a person's foot last and other foot characteristics in order to provide a more accurate fitting shoe based on the shape and volume of the person's foot. In addition, there is a need for a device and method that does not require the operator to undergo lengthy training procedures to learn how to use the device and method, nor require the operator to perform calculations.

### SUMMARY OF THE INVENTION

In accordance with the invention, there is provided a foot measuring device comprising a base having a rear end, a front end and a top surface; a heel abutment member attached to the base top surface for engaging a subject's heel; and a measurement scale affixed to the base top surface for providing a visual measurement of foot width, foot length, and foot last when the subject's heel is engaged with the heel abutment member; wherein the measurement scale comprises multiple spaced contour lines corresponding to categories of foot length, foot width, and foot last.

In another embodiment of the invention, there is provided a foot measuring device comprising a base having a rear end, a front end and a top surface; left and right heel abutment members positioned adjacently on the base top surface for engaging a subject's left and right heel respectively; a left foot measurement scale attached to the base top surface near the left heel abutment member; and a right foot measurement scale attached to the base top surface near the right heel abutment member; wherein the left and right foot measurement scales comprise multiple spaced contour lines corresponding to categories of foot length, foot width, and foot last for left and right feet.

In one embodiment, the food measuring device further comprises an arch height measurement member adjustable along the length of the base.

In another embodiment, the foot measuring device further comprises at least one support member attached to the base top surface; and a rail extending longitudinally along the base, the rail held above the base by the at least one support member; wherein the arch height measurement member is slidingly and pivotably engaged with the rail, the arch height measurement member moveable along the length of the rail and pivotable to a position on either side of the rail. The rail may be angled upwards from the rear end of the base to the front end of the base, and there may be an arch height indicator on the rail for displaying an arch height measurement.

In one embodiment, the foot measuring device comprises at least one lever having a first end pivotably engaged with the base top surface, the lever upwardly pivotable from the base for measuring metatarsophalangeal (MTP) joint flexibility. The base top surface may further include at least one depression adjacent the lever first end for engaging the ball of a foot. The base top surface may also include at least one protruding member adjacent the lever for engaging a space between a first and second toe.

In yet another embodiment, the foot measuring device further comprises a left and right lever, each lever having a first end pivotably engaged with the base top surface, each lever upwardly pivotable from the base for measuring left and right MTP joint flexibility. The levers may be pivotable from an angle of 0 degrees to 60 degrees with respect to the base. A flexibility indicator may display a MTP joint flexibility measurement.

The foot measuring device may further comprise a left depression in the base top surface adjacent the left lever first end for engaging the ball of a left foot; and a right depression in the base top surface adjacent the right lever first end for engaging the ball of a right foot. As well, there may be a left protruding member attached to the base top surface adjacent the left lever for engaging a space between a first and second left toe; and a right protruding member attached to the base top surface adjacent the right lever for engaging a space between a first and second right toe.

In another embodiment, the heel abutment member of the foot measuring device includes a window having at least one indicator for providing a visual measurement of the subject's heel position.

In another aspect of the invention, there is provided a method for measuring a subject's foot using the foot measuring device and comprising the steps of:
a. aligning the subject's left and right heels against the left and right heel abutment members;
b. visually determining left and right foot last, foot length and foot width using the left and right foot measurement scale;
c. rotating the foot contact member to a first side of the rail, perpendicular to the subject's first foot;
d. sliding the foot contact member backwards along the rail until it contacts a top surface of the subject's first foot;
e. determining a first foot arch height measurement based on the position of the foot contact member along the rail;
f. rotating the foot contact member to a second side of the rail, perpendicular to the subject's second foot and repeating steps d and e for the subject's second foot;
g. positioning the subject's first foot MTP joint on top the first end of the toe lever;
h. pivoting the toe lever upwards until resistance in the MTP joint is felt;
i. determining a first foot MTP joint flexibility measurement based on the angle of the toe lever in step i); and
j. repeating steps g)-i) for the subject's second foot MTP joint.

In another embodiment, the heel abutment member includes a window having at least one indicator, and the method further comprises the step of visually determining a heel position of the subject's foot using the window and indicators when the subject's heel is aligned against the heel abutment member.

### BRIEF DESCRIPTION OF THE DRAWINGS

The invention is described with reference to the accompanying figures in which:

FIG. 1 is diagram showing foot last and its effect on shoe fit;

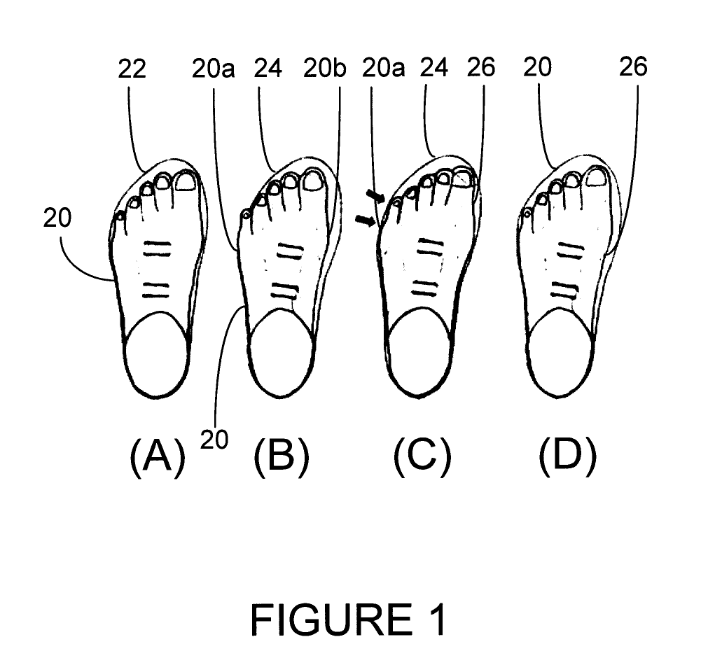

FIG. 2 is a perspective view of a foot measuring device in accordance with one embodiment of the invention;

FIG. 3 is a partial plan view of the foot measuring device showing a foot aligned with a heel cup and a chart for measuring a foot in accordance with one embodiment of the invention;

FIG. 4 is a plan view of the foot measuring device showing a Subject's foot positioned in the device in accordance with one embodiment of the invention;

FIG. 5 is a side view of the foot measuring device showing a Subject's foot and the components for measuring arch height in accordance with one embodiment of the invention;

FIG. 6 is a plan view of the foot measuring device showing a Subject's foot and the first step in measuring arch height in accordance with one embodiment of the invention;

FIG. 7 is a plan view of the foot measuring device showing a Subject's foot and the second step in measuring arch height in accordance with one embodiment of the invention;

FIG. 8 is a side view of the foot measuring device showing a Subject's foot and the second step in measuring arch height in accordance with one embodiment of the invention;

FIG. 9 is a plan view of the foot measuring device showing a Subject's foot and the first step in the operation of the MTP flexibility component in accordance with one embodiment of the invention;

FIG. 10 is a side view of the foot measuring device showing a user's foot and the second step in the operation of the MTP flexibility component in accordance with one embodiment of the invention;

FIG. 10A is a rear view of a heel abutment member with a standing pronation viewing window in accordance with one embodiment of the invention;

FIG. 11 is a flowchart of a method for measuring a foot in accordance with one embodiment of the invention;

FIG. 12 is a flowchart of a method for measuring arch height in accordance with one embodiment of the invention; and

FIG. 13 is a flowchart of a method for measuring metatarsophalangeal (MTP) joint flexibility in accordance with one embodiment of the invention.

### DETAILED DESCRIPTION OF THE INVENTION

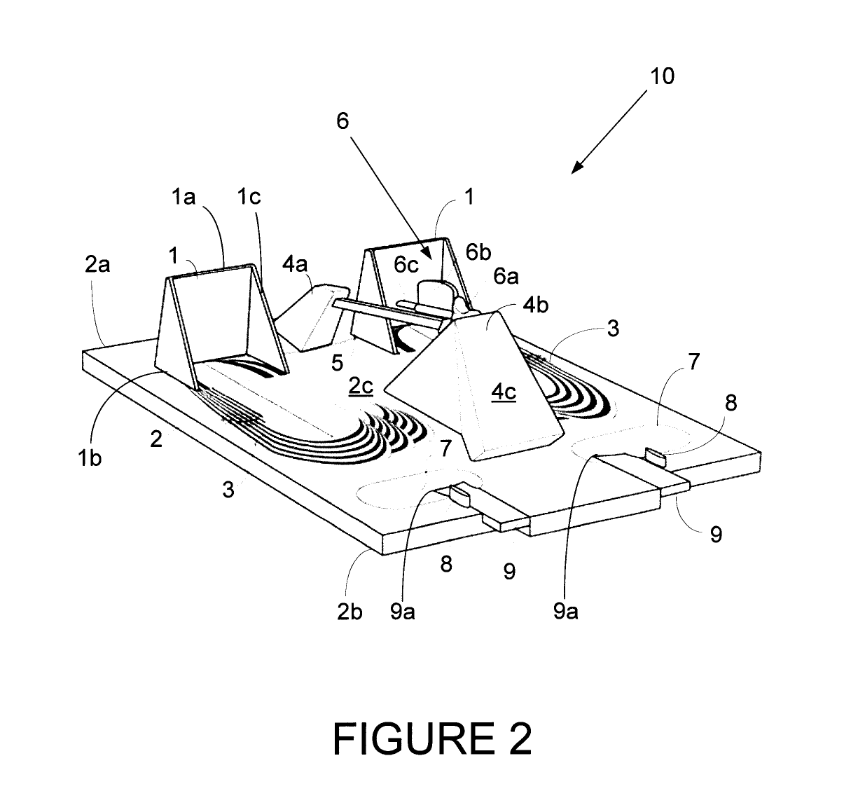

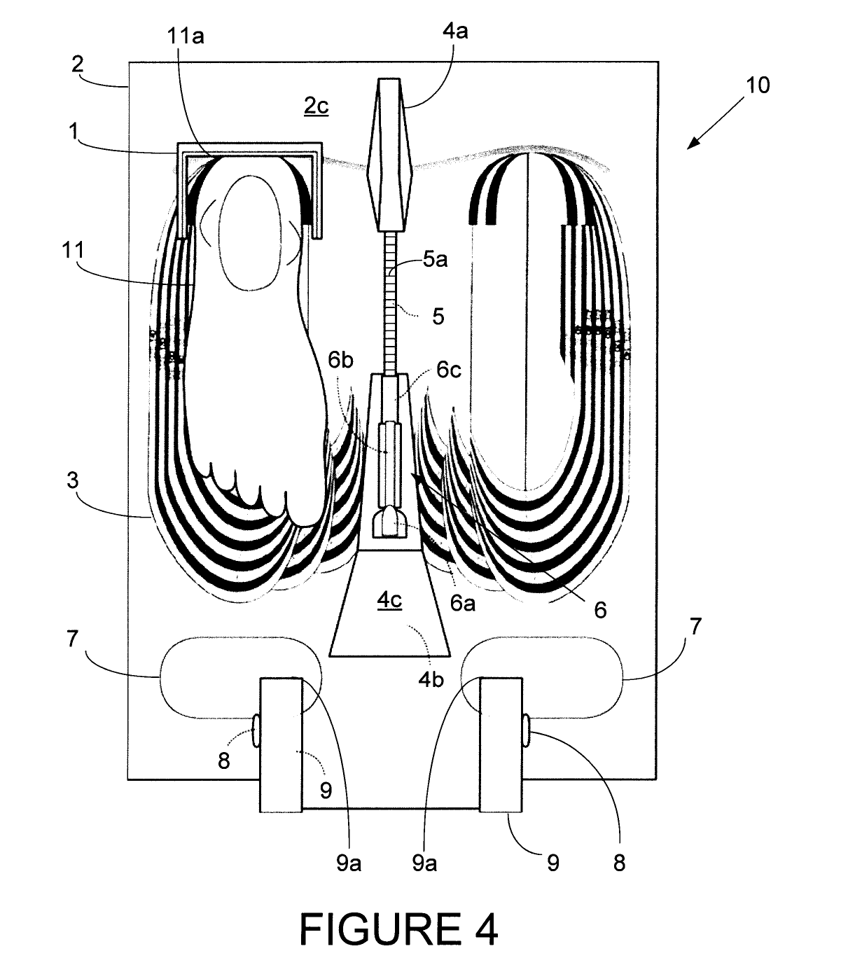

With reference to the figures, a foot measurement device and method are described. FIG. 2 illustrates an overview of all components included in a preferred embodiment of the foot measurement device 10. The device comprises a base 2 having a rear end 2a and a front end 2b with a flat non-slip top surface 2c wide enough to accommodate two feet and robust enough to support a standing person. A left and right foot size and alignment graphic 3 is printed or otherwise affixed to the base surface 2c. A left and right heel abutment member 1, a first and second support member 4a, 4b and a left and right toe separation member 8 are mounted to the top surface of the base 2 in the arrangement illustrated in FIG. 2. A left and right depression 7 for the placement of the ball of the foot are located towards the front end 2b of the base as illustrated in FIGS. 2 and 4. An inclined rail 5 is mounted between the first and second support member 4a, 4b to guide the movement of an arch height measurement member 6 which is free to translate along the length of the inclined rail 5.

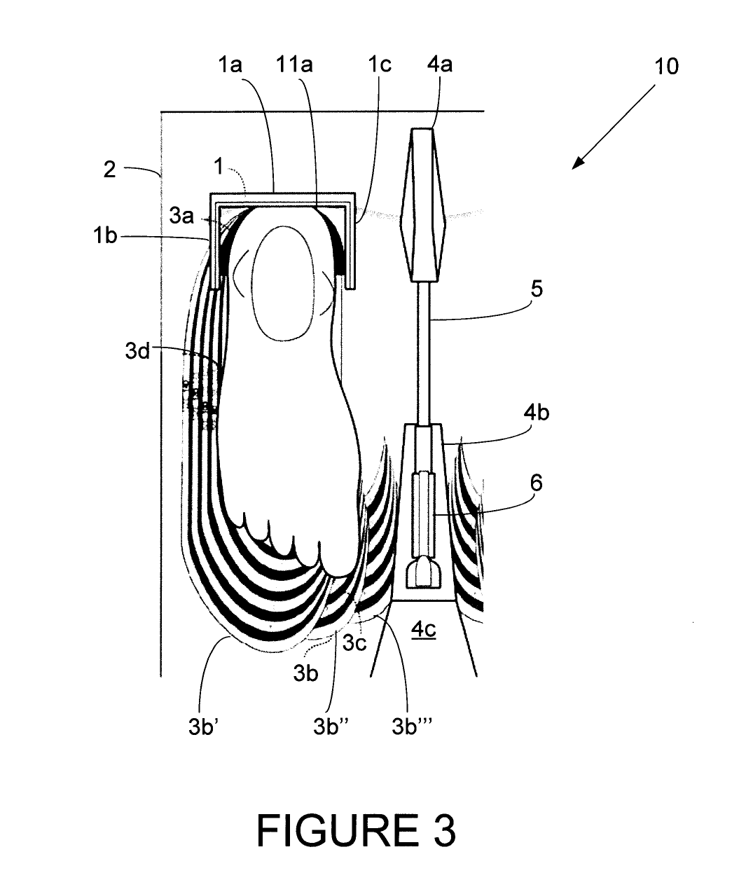

Details for the right foot size and alignment graphic 3 is shown in FIG. 3. The foot size and alignment graphic 3 includes components for heel alignment 3a, foot length 3c and foot width 3d that consist of contour lines that follow a general outline of a foot, and multiple contours are spaced apart by given distances which correspond to categorical differences in foot length and foot width. The graphic also includes a last curvature 3b component that corresponds to multiple sets of foot length graphics 3c which are translated and rotated to correspond with those curvatures present in footwear lasts, specifically a straight last 3b′, a semi-curved last 3b″ and a curved last 3b′″. The left side of the platform includes a corresponding left foot size and alignment graphic 3 that follows the general outline of a left foot, as shown in FIG. 4.

The first and second support member 4a, 4b are attached to the base top surface 2c and connected by the inclined rail 5 as shown in FIG. 2 and FIG. 4. The inclined rail slopes upwards towards the front end 2b of the base to allow for accurate measures of arch-height as a function of foot length (i.e. the longer the foot, the higher the arch, and thus the greater volume the foot occupies within a shoe). In the preferred embodiment, the inclined rail includes a measurement scale 5a for determining arch-height and the second support member 4b has an angled planar surface 4c which faces forward and away from the surface of the base, into which a measurement indicator (not shown), either electronic or mechanical, is integrated for the purpose of displaying arch height measurement information.

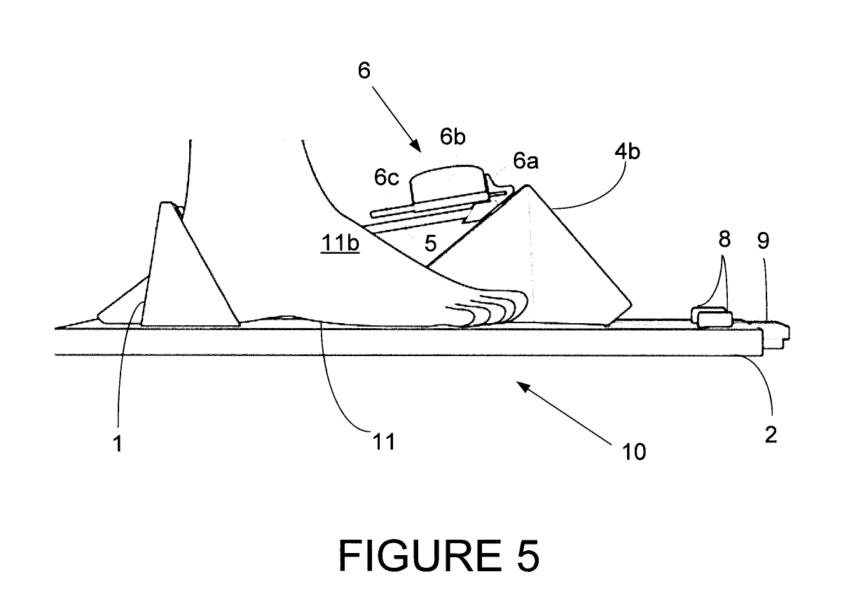

The arch height measurement member 6 is a component generally having the qualities of: free movement along the length of the inclined rail 5, and rotation of a foot contact member 6c to a position on either side of the inclined rail. As shown in FIG. 2, FIG. 4 and FIG. 5, in the preferred embodiment, the arch height measurement member 6 includes: a thumb rest 6a for the purpose of applying force to move the arch height measurement member along the inclined rail 5, a finger rest 6b for the purpose of rotating the arch height measurement member into a position on either side of the inclined rail and substantially perpendicular to the inclined rail 5, and the foot contact member 6c which consists in the general case of any cylindrical (cuboid, circular or ovoid) extension providing a singular, unique point of contact with the top of the foot.

Referring to FIG. 2, the left and right toe separation members 8 comprise in the general case of any thin, vertical extension from the surface of the base 2 which provides separation between the first and second toes, thus guiding the first toe to rest upon a lever 9. The lever 9 comprises in the general case of any structure integrated into the base which when manipulated with a nominal force causes extension of the first toe about the metatarsophalangeal (MTP) joint, with the remainder of the foot resting upon the base. In the preferred embodiment, the lever 9 consists of a flat rectangular block having a first end 9a that intersects the depression 7 and pivots upward from the depression. The lever may be freely pivotable or it may have a set end point angle that represents sufficient MTP joint flexibility, such as 60 degrees. In the general case, the measurement lever 9 will feature an indicator (not shown), electronic or mechanical, which displays a categorization of flexibility in the MTP joint as compared to a normal distribution of such a measurement.

Referring to FIG. 2 and FIG. 3, the left and right heel abutment members 1 in the general case comprise a vertical extending member from the surface of the base 2 for abutting a heel against in order to align a foot with the foot size and alignment graphic 3. The heel abutment member 1 may be formed as a “heel cup” having a rear wall 1a, and a first and second sidewall 1b, 1c for easier positioning of a heel.

### Further Embodiments

The foot measurement device may also include a standing pronation viewing window to provide a measure of hindfoot position. Previous research has shown that standing rearfoot position is a predictor of dynamic foot pronation biomechanics¹². Someone standing with his or her hindfoot collapsed inwards, would typically exhibit excessive foot pronation when walking or running. In contrast, someone standing with their hindfoot in an inverted or supinated position would not exhibit excessive pronation.

### The Measurement Procedure

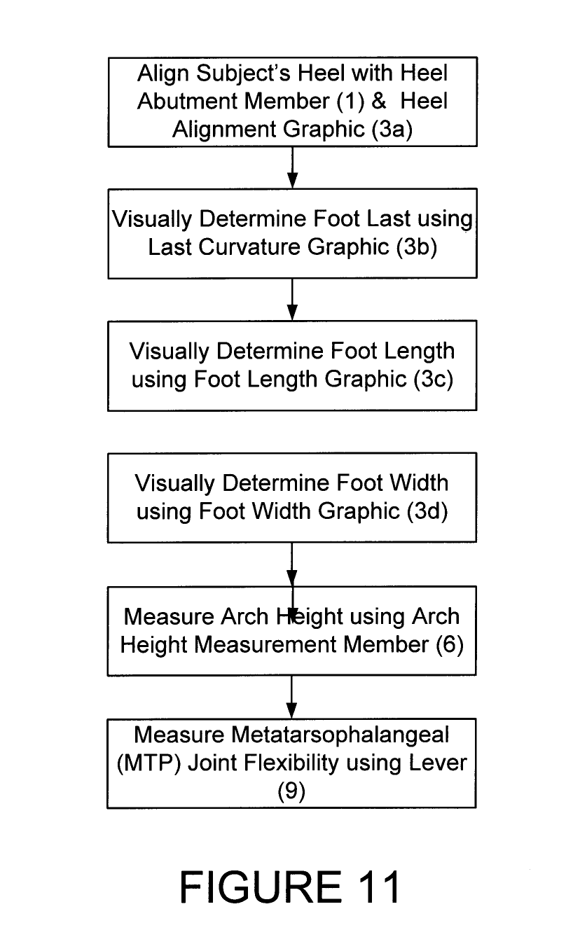

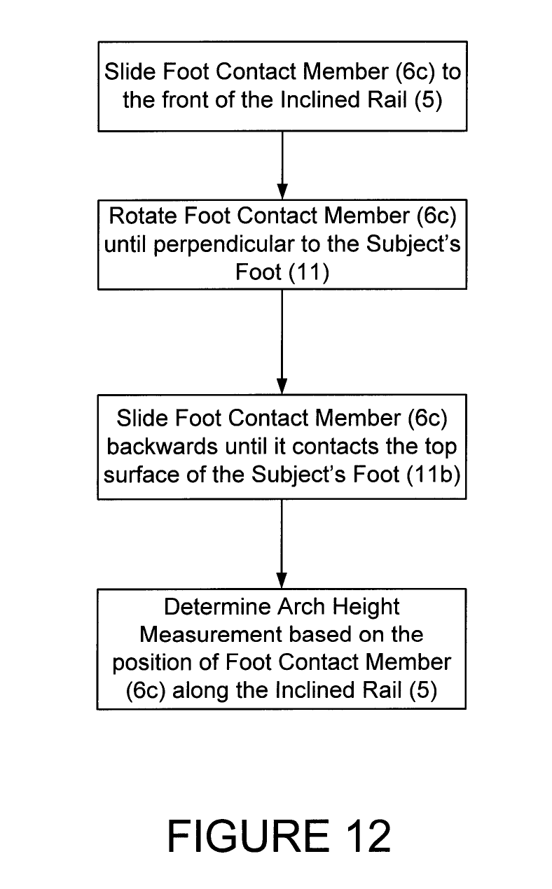

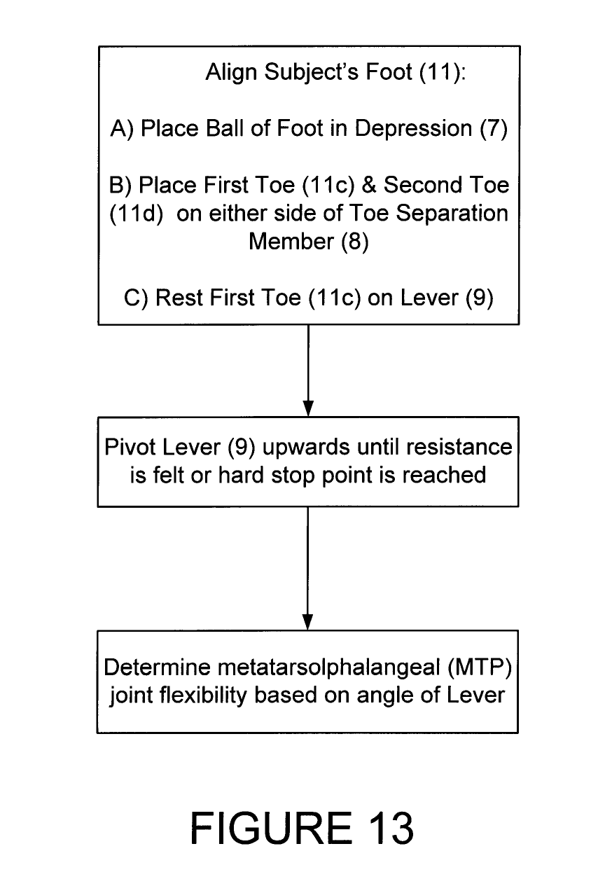

The operation of the invention for the purpose of fitting a shoe based on foot measurements will now be described with reference to the Figures. For the purpose of describing the operation of the invention, the person whose foot is being measured will be referred to as the Subject, and the person who is taking measurements using the invention will be referred to as the Operator. The description presented herein will utilize the preferred embodiment of the invention; however, all embodiments are claimed for the measurement procedure. Measurements are taken for both the left and right foot of the Subject. A general description of the measurement procedure is described in FIGS. 11, 12 and 13. Unless otherwise indicated, all measurements are taken in both a standing and seated position to allow the Operator to measure arch deformation and foot deformation, i.e. increases in the length and width of a Subject's foot when they move from a seated to a standing position.

### Measuring Foot Length, Width and Last

FIGS. 3 and 4 show a detailed plan view of the heel abutment member 1 and the size and alignment graphic 3a-d. The Subject, with bare feet, places each foot 11 on the surface of the device such that the Subject's heel 11a is in contact with the respective heel abutment member 1. The Operator aligns the Subject's foot 11 with the heel alignment graphic 3a, such that the curvatures of the Subject's heel 11a and the graphic 3a are concentric. The Operator determines which of the multiple sets of foot length graphics 3b′, 3b″, 3b′″ best corresponds to the Subject's foot last, and in the case of the preferred embodiment, the Subject's foot is designated as having a last typically used in the manufacture of footwear, either a straight last 3b′, a semi-curved last 3b″ or a curved last 3b′″.

Following the determination of the Subject's foot curvature, the Operator then observes which area of the foot length graphic 3c is still visible and not partially or entirely blocked by the Subject's foot. In the preferred embodiment of the invention, the Operator observes which banded area of the foot length graphic 3c can be seen in its entirety, and is therefore not blocked by the Subject's foot. This aforementioned area of the foot length graphic 3c is deemed to describe the length of the Subject's foot for fitting purposes, and in the case of the preferred embodiment, is assigned a categorical numerical value based on shoe sizing used in the manufacture of footwear for North American markets.

Following the determination of the Subject's foot length, the Operator then observes which area of the foot width graphic 3d is still visible and not partially or entirely blocked by the Subject's foot. In the preferred embodiment of the invention, the Operator observes which banded area of the foot width graphic 3d can be seen in its entirety, and is therefore not blocked by the Subject's foot. This aforementioned area of the foot width graphic 3d is deemed to describe the width of the Subject's foot for fitting purposes, and in the case of the preferred embodiment, is assigned a categorical value based on shoe sizing used in the manufacture of footwear for North American markets.

### Measuring Arch Height

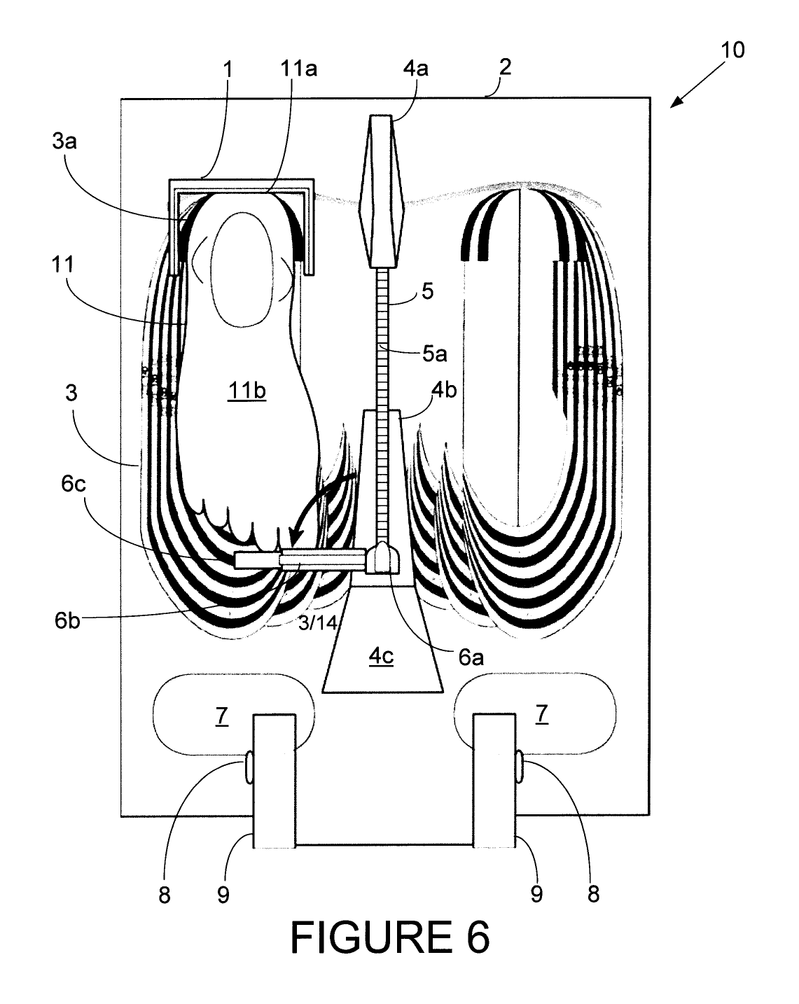

The operation of the invention for the purpose of measuring arch height is described with reference to FIGS. 6, 7 and 8. The Subject's heel 11a is aligned in the same way as for measuring foot length, last and width, with each heel in contact with the respective heel abutment member 1 and aligned with the heel alignment graphic 3a, such that the curvatures of the Subject's heel and the graphic 3a are concentric.

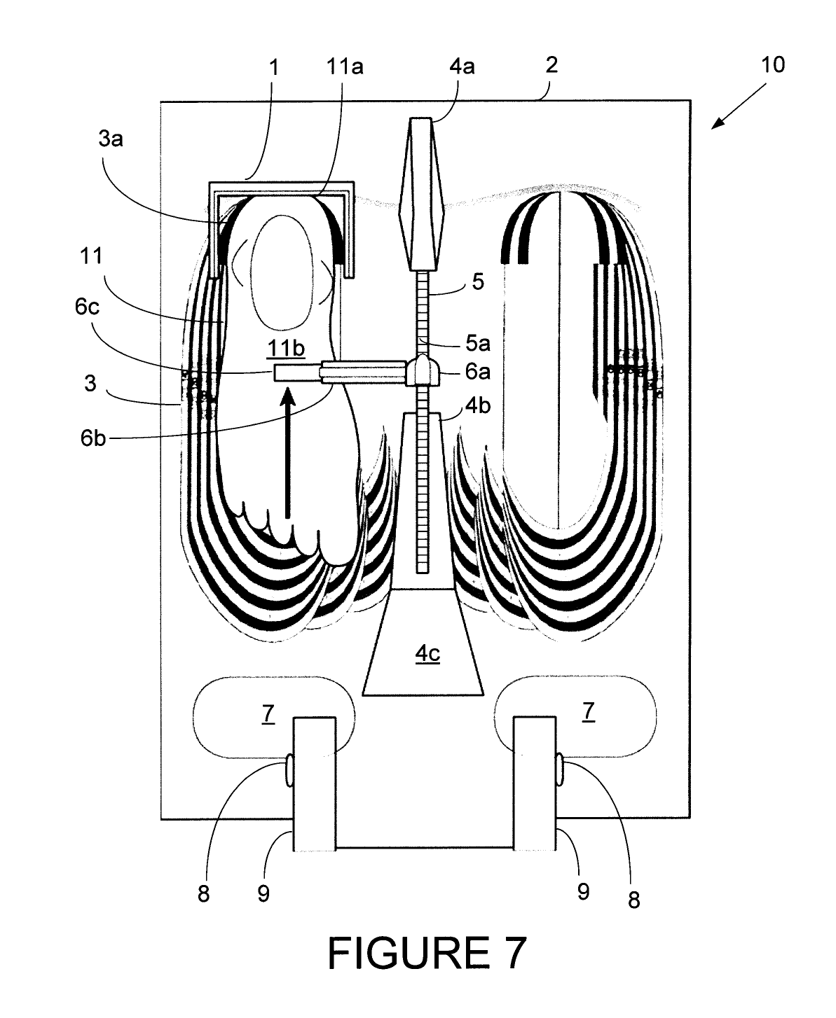

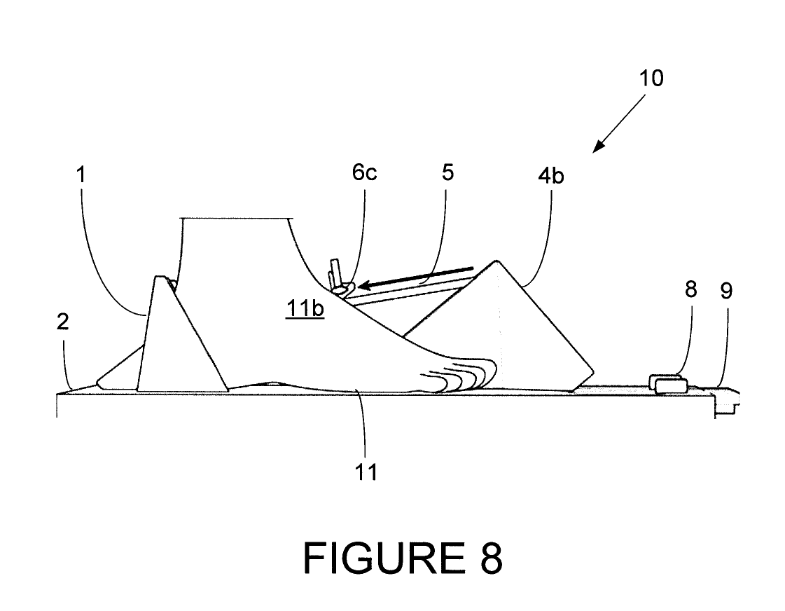

Following alignment of the Subject's heel, the Operator slides the foot contact member 6c to the second support member 4b end of the inclined rail 5 and rotates the foot contact member 6c to the side of the inclined rail that the Subject's foot is on, in a position relatively perpendicular to the Subject's foot, as shown in FIG. 6. The Operator then slides the foot contact member 6c along the inclined rail 5 towards the rear of the inclined rail, while keeping the foot contact member in the perpendicular position, until the foot contact member contacts a top surface 11b of the Subject's foot, as shown in FIG. 7 and FIG. 8. An arch height measurement is taken based on the position of the foot contact stylus along the inclined rail, preferably using a scale 5a located on the inclined rail. The arch height measurement can also be measured and displayed electronically.

The Operator repeats the arch height measurement procedure for the Subject's other foot by rotating the foot contact member 6c to the other side of the inclined rail 5 in a position perpendicular to the Subject's foot.

The arch-height scale is based on the same categorical numerical values for shoe sizing as foot length and foot width. For example, if a subject has a size 10 foot length, a normal arch height (based on research data) would be considered size 10. If the same subject has a low-arch, their arch-height measurement would be less than 10, such as 9, and if they have a high-arch, their arch-height measurement will be greater than 10, such as 11.

### Determining Foot Volume

In the preferred embodiment, the Operator determines a Subject's volumetric foot measurement based on foot width and/or arch height. Upon measuring a Subject's foot width and arch-height, the Subject's foot volume is determined based on whichever is greater: foot width or arch height. For example, if a subject has a size 10 foot length, size 10 foot width, and size 11 arch-height, the size 11 arch height would be used to determine foot volume. However if a subject has a size 10 foot length, size 11 foot width, and size 9 foot arch, the size 11 foot width would be used to determine foot volume.

### Measuring MTP Joint Flexibility

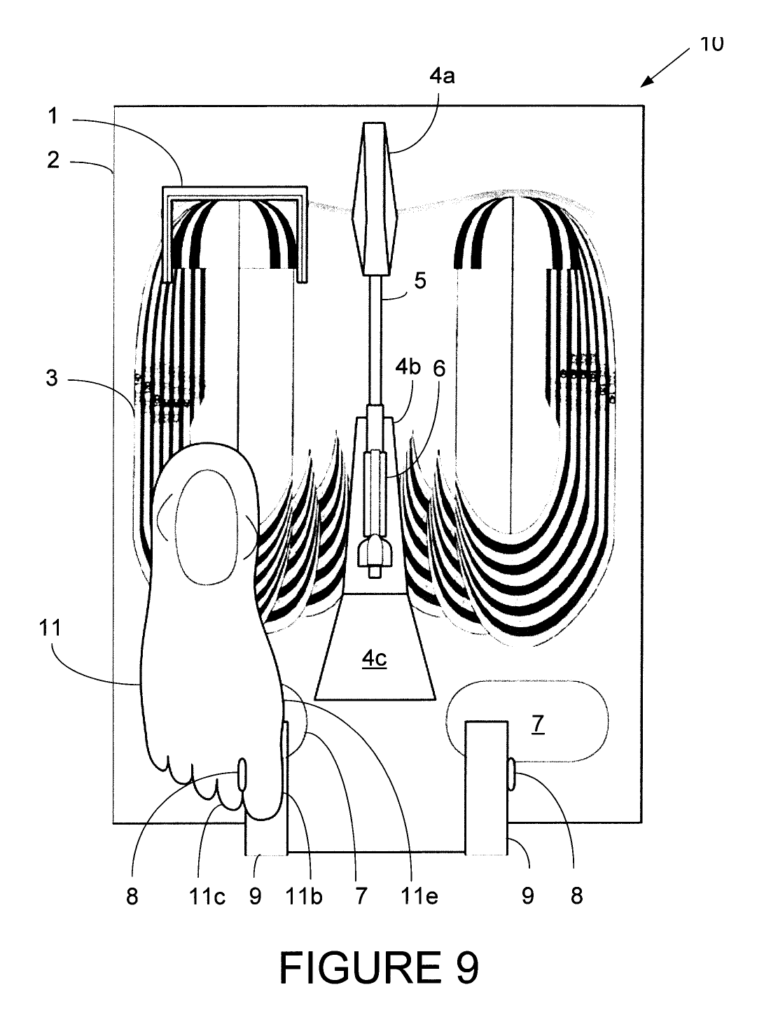

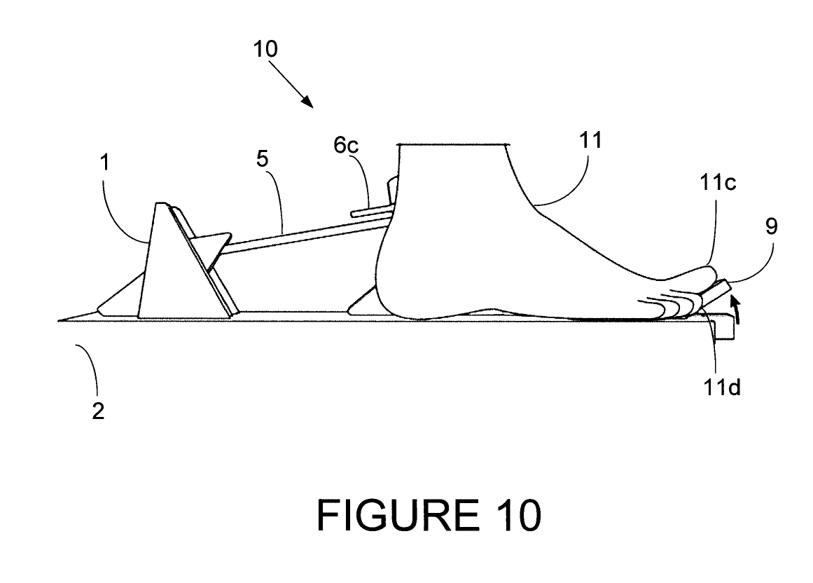

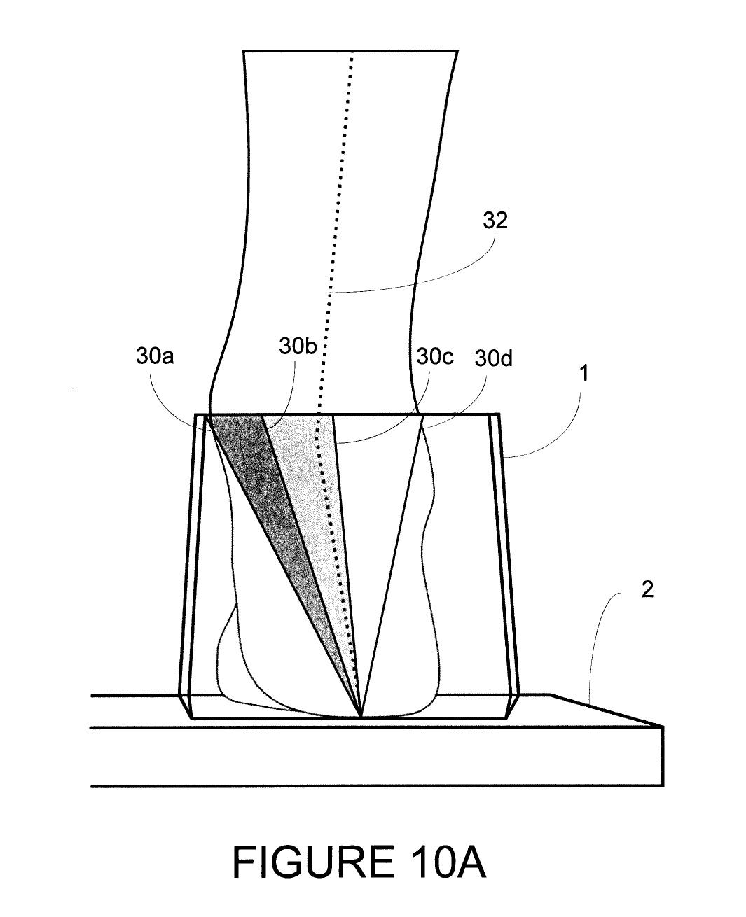

The measurement of MTP joint flexibility is described with reference to FIGS. 9 and 10. The Subject's foot 11 is positioned such that the ball of the foot 11e rests in the depression 7 in the base 2, the first and second toes 11c, 11d sit on either side of the toe separation member 8 with the first toe 11c resting on the lever 9, as illustrated in FIG. 9.

Following alignment of the Subject's foot, the Operator gently pivots the the lever 9 upwards as shown in FIG. 10 until either resistance is felt or the lever has reached its maximum cutoff angle. MTP joint flexibility is determined based on the angle of the lever, and in the preferred embodiment, an indicator on the device gives a reading of the angle and/or categorization of MTP joint flexibility.

The MTP joint flexibility measurement procedure is repeated for the Subject's other foot, using the depression, toe separation member and lever located on the other side of the base 2.

### Measuring Standing Pronation

FIG. 10A shows one embodiment of the invention wherein the heel abutment member 1 has a window 30 for determining pronation of the Subject's foot while standing. Following alignment of the Subject's heel with the heel abutment member, the Operator visually inspects the position of the Subject's heel by comparing a line 32 bisecting the heel of the Subject to lines 30a, 30b, 30c, 30d drawn on the window 30. The position of the line 32 relative to the lines 30a, 30b, 30c, 30d allows the Operator to predict foot dynamic movement (i.e. pronation) of the Subject during walking or running.

For example, if line 32 falls within lines 30b and 30c, the Operator can expect a typical foot dynamic from the Subject during walking or running. If line 32 falls within lines 30c and 30d, the Operator can expect reduced foot dynamic from the Subject during walking or running, and if line 32 falls within lines 30a and 30b, the Operator can expect excessive foot dynamic from the Subject during walking or running.

### Application of the Invention

By measuring foot length, foot width and foot last, the device and method provides three fundamental measurements that can be used to better fit the Subject's feet to specific shoes. It can also be used to measure and aid in the determination of arch height, foot volume, arch collapse as a function of foot length, atypical foot alignment, first ray valgus angulation as a measure of bunion development, hammer toe alignment, hallux MTP range of motion, and standing heel pronation. This information is important for preventing, diagnosing and treating certain foot conditions.

Although the present invention has been described and illustrated with respect to preferred embodiments and preferred uses thereof, it is not to be so limited since modifications and changes can be made therein which are within the full, intended scope of the invention as understood by those skilled in the art.

### REFERENCES

1) Goonetilleke R. S, Luximon A. Foot Flare and Foot Axis. Human Factors: The Journal of Human Factors and Ergonomics Society. 1999 41:596-607.
2) Krauss I, Grau S, Mauch M, Maiwald C, Horstmann T. Sex-related differences in foot shape. Ergonomics. 2008 November; 51(11):1693-709.
3) Wunderlich R E, Cavanagh P R. Gender differences in adult foot shape: implications for shoe design. Med Sci Sports Exerc. 2001 April; 33(4):605-11.
4) Manna I, Pradhan D, Ghosh S, Kar S K, Dhara P. A comparative study of foot dimension between adult male and female and evaluation of foot hazards due to using of footwear. J Physiol Anthropol Appl Human Sci. 2001 July; 20(4):241-6.
5) Swedler D I, Knapik J J, Grier T, Jones B H. Validity of plantar surface visual assessment as an estimate of foot arch height. Med Sci Sports Exerc. 2010 February; 42(2):375-80.
6) Butler RJ, Hamill J, Davis I. Effect of footwear on high and low arched runners' mechanics during a prolonged run. Gait Posture. 2007 July; 26(2):219-25.
7) Fields K B, Sykes J C, Walker K M, Jackson J C. Prevention of running injuries. Curr Sports Med Rep 2010 9(3):176-182.
8) McPoil T G, Cornwall M W, Medoff L, Vicenzino B, Fosberg K K, Hilz D. Arch height change during sit-to-stand: an alternative for the navicular drop test. J Foot Ankle Res. 2009 May 18; 2:17.
9) Jennings Weaver, L., Ferg, F. L. Therapeutic measurement and testing: the basics of ROM, MMT, posture and gait analysis. Delmar: Cengage Learning, 2010. p. 124.
10) Cornwall M W, McPoil T G, Fishco W D, O'Donnell D, Hunt L, Lane C. The influence of first ray mobility on forefoot plantar pressure and hindfoot kinematics during walking. Foot Ankle Int. 2006 July; 27(7):539-47.
11) Harrison S J, Cochrane L, Abboud R J, Leese G P. Do patients with diabetes wear shoes of the correct size? Int J Clin Pract. 2007 November; 61(11):1900-4.
12) Cornwall M W, McPoil T G. Relationship between static foot posture and foot mobility. J Foot Ankle Res. 2011 Jan. 18; 4:4.

1. A foot measuring device comprising:
a base having a rear end, a front end and a top surface;
a heel abutment member attached to the base top surface for engaging a subject's heel; and
a measurement scale affixed to the base top surface for providing a visual measurement of foot width, foot length, and foot last when the subject's heel is engaged with the heel abutment member;
wherein the measurement scale comprises multiple spaced contour lines corresponding to categories of foot length, foot width, and foot last.

2. A foot measuring device comprising:
a base having a rear end, a front end and a top surface;
left and right heel abutment members positioned adjacently on the base top surface for engaging a subject's left and right heel respectively;
a left foot measurement scale attached to the base top surface near the left heel abutment member; and
a right foot measurement scale attached to the base top surface near the right heel abutment member;
wherein the left and right foot measurement scales comprise multiple spaced contour lines corresponding to categories of foot length, foot width, and foot last for left and right feet.

3. The foot measuring device of claim 1 further comprising an arch height measurement member adjustable along the length of the base.

4. The foot measuring device of claim 3 further comprising:
at least one support member attached to the base top surface; and
a rail extending longitudinally along the base, the rail held above the base by the at least one support member;
wherein the arch height measurement member is slidingly and pivotably engaged with the rail, the arch height measurement member moveable along the length of the rail and pivotable to a position on either side of the rail.

5. The foot measuring device of claim 4 wherein the rail is angled upwards from the rear end of the base to the front end of the base.

6. The foot measuring device of claim 4 further comprising an arch height indicator on the rail for displaying an arch height measurement.

7. The foot measuring device of claim 1 further comprising at least one lever having a first end pivotably engaged with the base top surface, the lever upwardly pivotable from the base for measuring metatarsophalangeal (MTP) joint flexibility.

8. The foot measuring device of claim 7 further comprising at least one depression in the base top surface adjacent the lever first end for engaging the ball of a foot.

9. The foot measuring device of claim 7 further comprising at least one protruding member attached to the base top surface adjacent the lever for engaging a space between a first and second toe.

10. The foot measuring device of claim 2 further comprising a left and right lever, each lever having a first end pivotably engaged with the base top surface, each lever upwardly pivotable from the base for measuring MTP joint flexibility.

11. The foot measuring device of claim 10 further comprising:
a left depression in the base top surface adjacent the left lever first end for engaging the ball of a left foot; and
a right depression in the base top surface adjacent the right lever first end for engaging the ball of a right foot.

12. The foot measuring device of claim 10 further comprising:
a left protruding member attached to the base top surface adjacent the left lever for engaging a space between a first and second left toe; and
a right protruding member attached to the base top surface adjacent the right lever for engaging a space between a first and second right toe.

13. The foot measuring device of claim 7 wherein the lever is pivotable from an angle of 0 degrees to 60 degrees with respect to the base.

14. The foot measuring device of claim 7 further comprising a flexibility indicator for displaying a MTP joint flexibility measurement.

15. The foot measuring device of claim 1 wherein the heel abutment member includes a window having at least one indicator for providing a visual measurement of the subject's heel position.

16. A foot measuring device comprising:
a base having a rear end, a front end and a top surface;
left and right heel abutment members positioned adjacently on the base top surface for engaging a subject's left and right heel respectively;
a left foot measurement scale attached to the base top surface near the left heel abutment member and a right foot measurement scale attached to the base top surface near the right heel abutment member, wherein the left and right foot measurement scales comprise multiple spaced contour lines corresponding to categories of foot length, foot width, and foot last for left and right feet;
at least one support member attached to the base top surface;
a rail extending longitudinally between the left and right foot measurement scale, the rail held above the base by the at least one support member;
a foot arch abutment member slidingly and pivotably engaged with the rail, the foot arch abutment member moveable along the length of the rail and pivotable between a first and second position on either side of the inclined rail; and
a toe lever having a first end pivotably engaged with the base top surface, the toe lever upwardly pivotable from the base.

17. A method for measuring a subject's foot using the foot measurement device of claim 12 comprising the steps of:
a) aligning the subject's left and right heels against the left and right heel abutment members;
b) visually determining left and right foot last, foot length and foot width using the left and right foot measurement scale;
c) rotating the foot contact member to a first side of the rail, perpendicular to the subject's first foot;
d) sliding the foot contact member backwards along the rail until it contacts a top surface of the subject's first foot;
e) determining a first foot arch height measurement based on the position of the foot contact member along the rail;
f) rotating the foot contact member to a second side of the rail, perpendicular to the subject's second foot and repeating steps d and e for the subject's second foot;
g) positioning the subject's first foot MTP joint on top the first end of the toe lever;
h) pivoting the toe lever upwards until resistance in the MTP joint is felt;
i) determining a first foot MTP joint flexibility measurement based on the angle of the toe lever in step i); and
j) repeating steps g)-i) for the subject's second foot MTP joint.

18. The method of claim 17, wherein the heel abutment member includes a window having at least one indicator, and the method further comprises the step of visually determining a heel position of the subject's foot using the window and indicators when the subject's heel is aligned against the heel abutment member.

19. The foot measuring device of claim 2 further comprising:
at least one support member attached to the base top surface;
a rail extending longitudinally along the base, the rail held above the base by the at least one support member; and
an arch height measurement member slidingly and pivotably engaged with the rail, the arch height measurement member moveable long the length of the rail and pivotable to a position on either side of the rail for measuring arch height.

20. The foot measuring device of claim 19 wherein the rail is angled upwards from the rear end of the base to the front end of the base, and the rail includes in arch height indicator for displaying arch height measurement.
